(19) United States
(12) Patent Application Publication
(10) Pub. No.: US 2025/0057454 A1
(43) Pub. Date: Feb. 20, 2025

(54) MENTAL STRESS CLASSIFICATION APPARATUS AND METHOD USING ENSEMBLE MODEL

(71) Applicant: Industry-Academic Cooperation Foundation, Chosun University, Gwangju (KR)

(72) Inventors: Youn Tae KIM, Daejeon (KR); Jae Hyo JUNG, Gwangju (KR); Si Ho SHIN, Gwangju (KR)

(21) Appl. No.: 18/807,298

(22) Filed: Aug. 16, 2024

(30) Foreign Application Priority Data
Aug. 16, 2023 (KR) 10-2023-0107004

(51) Int. Cl. A61B 5/16; A61B 5/00; A61B 5/346; G06N 7/01; G06N 20/10; G06N 20/20

(52) U.S. Cl. CPC A61B 5/165; A61B 5/346; A61B 5/725; A61B 5/7264; G06N 7/01; G06N 20/10; G06N 20/20

(57) ABSTRACT

A mental stress classification apparatus using an ensemble model includes a processor and a storage medium on which one or more programs configured to be executable by the processor are recorded. The processor is configured to extract a feature vector of an electrocardiogram (ECG) signal and classify the extracted feature vector using the ensemble model, wherein the ensemble model is a mixture model of a support vector machine and a naive Bayes.

### BACKGROUND

### 1. Field

The present disclosure relates to a mental stress classification apparatus and method using an ensemble model.

### 2. Description of Related Art

Recently, mental illnesses, caused by factors, such as stress, have emerged as a social problem in modern society. Stress refers to a physical and mental state of a body caused by external factors affecting the body. Excessive mental stress may cause chronic diseases, such as headaches, high blood pressure, and skin diseases, so it is necessary to prevent and treat this condition.

In general, stress assessment determines an individual's stress state through questionnaire-based stress analysis. However, this approach cannot determine the exact stress state due to individual variations, and people may be reluctant to answer questions, so the reliability of stress assessment may be low. In order to accurately determine the state of stress, research has been conducted to measure stress using biological signals and then determine the state of stress using machine learning.

### SUMMARY

An aspect of the present disclosure is to provide a mental stress classification apparatus and method using an ensemble model capable of more accurately classifying stress levels according to emotional states.

According to an aspect of the present disclosure, a mental stress classification apparatus using an ensemble model includes a processor and a storage medium on which one or more programs configured to be executable by the processor are recorded, wherein the processor is configured to extract a feature vector of an electrocardiogram (ECG) signal and classify the extracted feature vector using the ensemble model, wherein the ensemble model is a mixture model of a support vector machine and a naive Bayes.

According to another aspect of the present disclosure, a mental stress classification method using an ensemble model includes an extraction operation of extracting a feature vector of an electrocardiogram (ECG) signal and a classification operation of classifying the extracted feature vector using the ensemble model, wherein the ensemble model is a mixture model of a support vector machine and a naive Bayes.

According to another aspect of the present disclosure, there is provided a computer-readable storage medium on which a program for executing the mental stress classification method on a computer is recorded.

### BRIEF DESCRIPTION OF DRAWINGS

The above and other aspects, features, and advantages of the present disclosure will be more clearly understood from the following detailed description, taken in conjunction with the accompanying drawings, in which:

FIG. 1 is a diagram illustrating an entire system including a mental stress classification apparatus using an ensemble model according to an embodiment of the present disclosure.

FIGS. 2A, 2B and 2C are diagrams illustrating various electrocardiogram signals and decision boundaries and contours stored in a database according to an embodiment of the present disclosure.

FIG. 3 is a diagram illustrating classification accuracy using 10-fold cross-validation.

FIG. 4 is a diagram illustrating classification accuracy using a confusion matrix.

FIGS. 5A, 5B and 5C are diagrams illustrating classification accuracy using an ROC curve.

FIG. 6 is a flowchart illustrating a mental stress classification method using an ensemble model according to an embodiment of the present disclosure.

FIG. 7 is a block diagram of a computing device capable of fully or partially implementing a mental stress classification apparatus using an ensemble model according to an embodiment of the present disclosure.

### DETAILED DESCRIPTION

Hereinafter, embodiments of the present disclosure are described with reference to the accompanying drawings. The following description is provided to aid in the comprehensive understanding of methods, devices, and/or systems disclosed in the particularities. However, the following description is merely exemplary and not provided to limit the present disclosure.

In the following description of the present disclosure, a detailed description of known functions and configurations incorporated herein will be omitted when the subject matter of the present disclosure would be rendered unclear. The terms used in the present specification are defined in consideration of functions used in the present disclosure, and may be changed according to the intent or conventionally used methods of clients, operators, and users. Accordingly, definitions of the terms should be understood on the basis of the entire description of the present specification. Terms used in the following description are merely provided to describe embodiments of the present disclosure and are not intended to be limiting of the inventive concept. As used herein, the singular forms “a,” “an” and “the” are intended to include the plural forms as well, unless the context clearly indicates otherwise. It will be further understood that the terms “comprises” or “has” when used in this specification, specify the presence of stated features, integers, steps, operations, elements, or a portion or combination thereof, but do not preclude the presence or addition of one or more other features, integers, steps, operations, elements, or a portion or combination thereof.

Hereinafter, specific embodiments of the present disclosure are described with reference to the drawings.

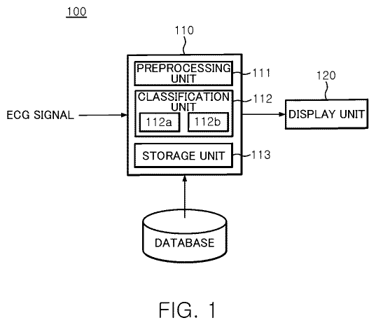

FIG. 1 is a diagram illustrating the entire system including a mental stress classification apparatus using an ensemble model according to an embodiment of the present disclosure.

First, an entire system 100 may include a mental stress classification apparatus 110 and a display unit 120 and may further include a database 10 storing electrocardiogram (ECG) signals for learning. The mental stress classification apparatus 110 may include a preprocessing unit 111, a classification unit 112, and a storage unit 113, and the classification unit 112 may include a support vector machine 112a and a naive Bayes 112b.

Specifically, the preprocessing unit 111 of the mental stress classification apparatus 110 may remove noise by low-pass filtering an ECG signal. The low-pass filtered ECG signal may be transmitted to the classification unit 112. According to an embodiment of the present disclosure, the preprocessing unit 111 may be a Butterworth low-pass filter.

When measuring ECG signals, noise may occur due to various factors. Various noises make it difficult to extract accurate feature points when analyzing ECG signals and significantly reduce accuracy. To solve this, a low-pass filter was used. Low-pass filters include Butterworth low-pass filter and Chebyshev low-pass filter. However, the Chebyshev low-pass filter is disadvantageous in that ripples may occur in a pass band compared to the Butterworth low-pass filter, which may cause noise due to signal distortion. The Butterworth low-pass filter is a flat filter that does not cause ripples in the pass band and has the characteristic of attenuating a high-frequency band. Therefore, by outputting clearer ECG signals than other filters, stress classification accuracy may increase. Noise was reduced using a low-pass filter in which a sampling frequency of the ECG signal was 330 Hz and a cutoff frequency was 120 Hz.

The classification unit 112 may extract feature vectors of the ECG signal and classify the extracted feature vectors using an ensemble model. The classification unit 112 includes an ensemble model, and the ensemble model may be a mixture model of the support vector machine 112a and the naive Bayes 112b.

According to an embodiment of the present disclosure, the aforementioned feature vector may include at least one of an R-S peak, R-R interval, and Q-T interval of the ECG signal, and the classification unit 112 classifies the ECG signals based on each feature vector.

Specifically, the support vector machine 112a described above may be a previously trained model that classifies each feature vector into one of multi-classes using a decision boundary.

As is known, the decision boundary may be obtained using Equation 1 and Equation 2 below through a learning process. The decision boundary is also referred to as a hyperplane.

$$[ Equation 1 ]$$

$$W \cdot {X}_{1} + B = \pm 1 , c = 1 , … , N$$

$$[ Equation 2 ]$$

$$Min \frac{1}{2} {‖ w ‖}^{2}$$

In Equation 1 and Equation 2 above, c denotes the number of classes, X1 denotes the decision boundary, W denotes a vector perpendicular to the decision boundary, B denotes a bias, and Min denotes a margin.

Meanwhile, Table 1 shows multi-classes according to an embodiment of the present disclosure, and these multi-classes are based on various ECG signals stored in the database 10.

TABLE 1

| | | | Stress | | Stress | | Stress |
|---|---|---|---|---|---|---|---|
| Class | R-Speak (mv) | | Level | R-R Interval(s) | Level | Q-T Interval(s) | Level |
| Class1 | 0~1.69 | (mv) | 1~1.9 | 0.85~0.95(s) | 1~1.9 | 1.3~1.44(s) | 1~1.9 |
| Class2 | 1.7~2.5 | mv) | 2~2.9 | 0.76~0.759(s) | 2~2.9 | 1.191~1.29(s) | 2~2.9 |
| Class3 | 2.51~3.3 | (mv) | 3~3.9 | 0.57~0.649(s) | 3~3.9 | 0.981~1.19(s) | 3~3.9 |
| Class4 | 3.31~4 | mv) | 4~4.9 | 0.85~0.95(s) | 4~4.9 | 0.85~0.98(s) | 4~4.9 |

Specifically, as shown in Table 1, in class 1, ECG signals according to test subject's emotions may be various ECG signals that measure an emotional state by looking at landscape photos (Picture Test, PI). Class 2 may be various ECG signals that measure an emotional state after listening to classical music (Music Test, MU). Class 3 may be various ECG signals that measure an emotional state after matching colors through visual stimulation (Stroop Test, ST). Class 4 may include various ECG signals that measure an emotional state after calculating the four arithmetic operations (Math Test, MA).

That is, Table 1 labels various ECG signals stored in the database 10, which will be described below, for learning, with respect to respective feature vectors (R-S peak, R-R interval, Q-T interval), and the stress level increases from class 1 to class 4.

Meanwhile, the naive Bayes 112b may be a previously trained model that classifies a stress index for a feature vector based on a preset contour plot in a class classified by a decision boundary obtained through a support vector machine.

As is generally known, naive Bayes is a statistical classification method based on conditional probability which calculates the probability that data, that is, a feature vector, belongs to each class. In the classes classified by the decision boundary using the generally known Equation 3, a stress index for the feature vector may be classified.

$$[ Equation 3 ]$$

$$p ( B \vert A ) = \frac{p ( A , B )}{P ( A )} , p ( A , B ) = p ( B \vert A ) P ( A )$$

At this time, a preset contour plot may be used. The aforementioned contour plot may be obtained using a probability density function of a normal distribution for feature vectors belonging to each multi-class based on the ECG signals stored in the database 10 through a learning process.

Meanwhile, the aforementioned database 10 is a cognitive load affect and stress (CLAS) database storing various ECG signals according to the test subject's emotions and may be used as training data for learning.

Specifically, the ECG signals according to the test subject's emotions may include various ECG signals (Picture Test, PI) of measuring an emotional state by looking at landscape photos, various ECG signals (Music Test, MU) of measuring an emotional state after listening to classical music, various ECG signals (Stroop Test, ST) of measuring an emotional state after matching colors through visual stimulation, and various ECG signals (Math Test, MA) of measuring an emotional state after calculating the four arithmetic operations.

Meanwhile, the storage unit 113 may store various programs to implement the functions performed by the classification unit 112 described above.

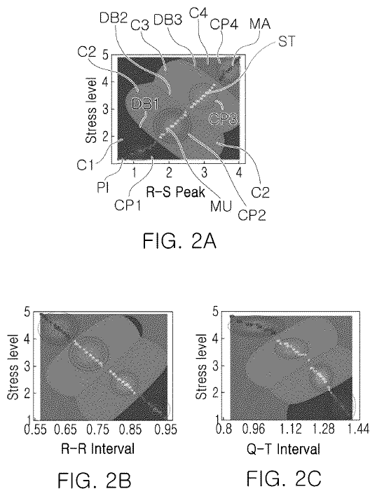

In addition, the display unit 120 may output a classification result from the mental stress classification apparatus 110. In addition, the display unit 120 may display the form of FIGS. 2A, 2B and 2C as described below.

Meanwhile, FIGS. 2A, 2B, 2C are diagrams illustrating various ECG signals, decision boundaries, and contour lines stored in the database according to an embodiment of the present disclosure. FIG. 2A is for an R-S peak value among the feature vectors of the ECG signals stored in the database 10, FIG. 2B is for an R-R interval among the feature vectors of the ECG signal stored in the database 10, and FIG. 2C is for a Q-T interval among the feature vectors of the ECG signals stored in the database 10. Since FIG. 2A, 2B, 2C are similar, FIG. 2A is mainly described to simplify the disclosure.

Referring to FIG. 2A, various feature vectors PI, MU, ST, and MA of the ECG signals stored in the database 10 are indicated as points in the figures. The points include feature vectors PI belonging to the label of class 1 (C1), feature vectors MU belonging to the label of class 2 (C2), feature vectors ST belonging to the label of class 3 (C3), and feature vectors MA belonging to the label of class 4 (C4). In the drawing, classes can be expressed in different colors.

Once learning is complete, the decision boundaries DB1 to DB3 may be obtained, and a decision boundary DB1 separating class 1 (C1) and class 2 (C2), a decision boundary DB2 separating class 2 (C2) and class 3 (C3), and a decision boundary DB3 separating class 3 (C3) and class 4 (C4) may be obtained. Thereafter, when a new ECG signal is input, the classification unit 112 may classify the R-S peak value of the input ECG signal into one of the multi-classes C1 to C4 based on the aforementioned decision boundaries DB1 to DB3.

Meanwhile, contour plots CP1 to CP4 may be set within each of the classes C1 to C4. The contour plot may be obtained using the probability density function of the normal distribution for each feature vector. That is, in the case of class 1 (C1), the contour plot may be obtained using the probability density function of the normal distribution for the R-S peak values PI belonging to class 1 (C1), in the case of class 2 (C2), the contour plot may be obtained using the probability density function of the normal distribution for the R-S peak values MU belonging to class 2 (C2), in the case of class 3 (C3), the contour plot is obtained using the probability density function of the normal distribution for the R-S peak values ST belonging to class 3 (C3), and in the case of class 4 (C4), the contour plot is obtained using the probability density function of the normal distribution for the R-S peak values MA belonging to class 4 (C4). As shown, these contours may be displayed in different colors as they are away from an average value. Thereafter, when a new ECG signal is input, the classification unit 112 may classify a stress index for the R-S peak value, which is a feature vector, based on the contour plot. For example, in Table 1, the stress index for the R-S peak value may be classified as any value between 1 and 4.9.

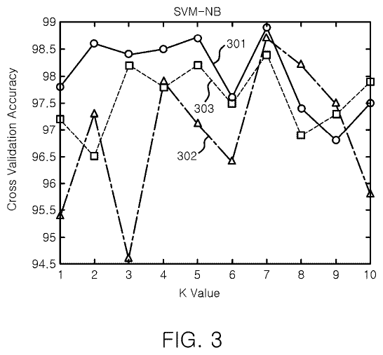

Meanwhile, FIG. 3 is a diagram illustrating classification accuracy using 10-fold cross-validation. In FIG. 3, reference numeral 301 denotes the R-S peak value, reference numeral 302 denotes the R-R interval, and reference numeral 303 denotes the Q-T interval.

In K-fold cross validation, data is divided into k groups, one of the groups is extracted and used as a test set, and the remaining K−1 groups are used as a training set. Repeating K times, each test obtains one classification accuracy, and then obtains an average K result to obtain the final performance of the classification.

In the present disclosure, the performance of an ensemble model combining a support vector machine and naive Bayes is demonstrated using 10-fold cross-validation. As a result of the classification, overfitting may be prevented by illustrating accuracies of 98.9%, 98.7%, and 98.4% at 7 folds.

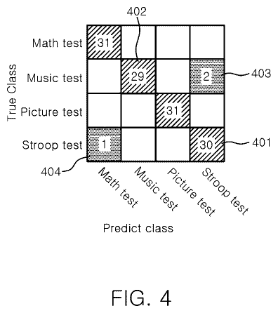

Meanwhile, FIG. 4 is a diagram illustrating classification accuracy using a confusion matrix.

In FIG. 4, reference numeral 401 (hatched) denotes true positive (TP). Reference numeral 402 is a result value of a designed classifier correctly classifying the data. Reference numeral 403 denotes false positive (FP). Reference numeral 404 is a result value of the designed classifier incorrectly classifying music video-related data as Stroop Test data. Therefore, data related to Math Test and Picture Test are classified more correctly than data related to Stroop Test and Music video.

Table 2 below evaluates stress classification performance using the confusion matrix.

TABLE 2

| | R-S Peak(mv) | | | R-R Interval(s) | | | Q-T Interval(s) | | | Mean | | |
|---|---|---|---|---|---|---|---|---|---|---|---|---|
| Model | AC(%) | AP(%) | AR(%) | AC(%) | AP(%) | AR(%) | AC(%) | AP(%) | AR(%) | AC(%) | AP(%) | AR(%) |
| SVM | 97.6 | 96 | 95.4 | 94.4 | 93 | 92.8 | 96.8 | 95.4 | 94.4 | 96.3 | 94.8 | 94.2 |
| NB | 96.8 | 95.7 | 94.1 | 93.5 | 92.8 | 92.2 | 95.2 | 94.8 | 93.6 | 95.2 | 94.3 | 92.3 |
| SVM | 98.4 | 98.2 | 98.1 | 96.8 | 96.7 | 96.7 | 97.6 | 97.5 | 97.3 | 97.6 | 97.5 | 97.4 |
| and NB | | | | | | | | | | | | |

In the present disclosure, Table 2 is used to show values of average accuracy, average precision, and average recall rate of the stress classification model. Equation 4 below means accuracy, which is the probability of correctly classifying four emotional states. Total Data set refers to the total number of pieces of ECG data. Using the stress classification model combining the support vector machine and the naive Bayes, the average accuracy of R-S peak, R-R interval, and Q-T interval was 97.6%.

$$[ Equation 4 ]$$

$$Accuracy = TP / ( TotalDataset )$$

Also, Equation 5 below means precision. For example, it is the probability that an algorithm is correctly classified as Picture Test during Picture Test. In a similar manner, after precision for the four emotional states was calculated and the average precision was presented. Using a stress classification model combining the support vector machine and the naive Bayes, the average precision according to R-S peak, R-R interval, and Q-T interval was 97.5%.

$$[ Equation 5 ]$$

$$Precison = TP / ( TP + FP )$$

Meanwhile, Equation 6 below means a recall rate. This is the probability that the algorithm is correctly classified as Picture Test among the data predicted by Picture Test. In a similar manner, the recall rates for the four emotional states were calculated and an average recall rate was shown. Using the stress classification model combining the support vector machine and the naive Bayes, the average recall rate according to R-S peak, R-R interval, and Q-T interval was 97.4%.

$$[ Equation 6 ]$$

$$Recall = TP / ( TP + FN )$$

In the present disclosure, the average accuracy of the stress classification model using the support vector machine was 96.3%. In addition, the performance of the stress classification model was evaluated by combining the naive Bayes model with the support vector machine. As a result, the average accuracy of the stress classification model combining the support vector machine and the naive Bayes was 97.6%. These results show that accuracy improved by 8.7% compared to the stress classification model using the existing CLAS Dataset and that stress classification using four levels may more accurately classify emotional states than stress classification using two levels.

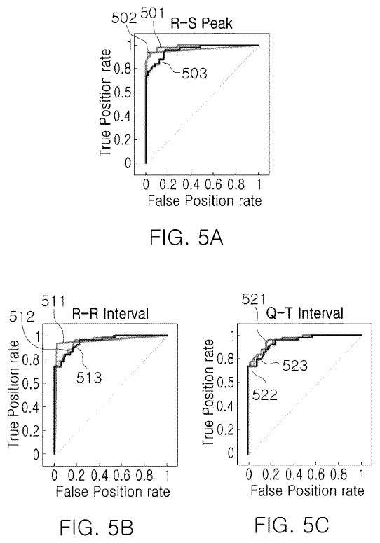

Lastly, FIGS. 5A, 5B, 5C are diagrams illustrating classification accuracy using an ROC curve. In FIGS. 5A, 5B, 5C, reference numerals 501, 511 and 521 are ROC curves of the stress classification model combining the support vector machine and the naive Bayes, reference numerals 502, 512, 522 are ROC curves of the stress classification model based on the support vector machine, and reference numerals 503 are ROC curves of the stress classification model based on the naive Bayes.

ROC curve analysis is a curve drawn with a true positive rate on the Y-axis and a false positive rate on the X-axis of the tested values. The performance of the stress classification model is evaluated using an area under the curve (AUC), which is the area under the curve in the graph of ROC Curve. Depending on the AUC value, the stress classification performance may be classified as low accuracy (AUC≤0.7), medium accuracy (0.7<AUC≤0.9), and high accuracy (0.9<AUC<1). Table 3 below evaluates stress classification performance using the ROC curve.

TABLE 3

| AUC(ROC | R-S | R-R | Q-T | Mean |
|---|---|---|---|---|
| Curve) | Peak(mv) | Interval(s) | Interval(s) | AUC(%) |
| SVM | 97.5% | 96.8% | 96.2% | 96.8% |
| NB | 96.2% | 95.1% | 94.9% | 95.4% |
| SVM and NB | 98.2% | 98% | 97.6% | 97.9% |

As shown in Table 3, the performance was compared with the existing stress classification model using the AUC value of the ROC Curve. As a result, the average AUC according to the stress classification model combining the support vector machine and the naive Bayes was 97.9%, which was 1.1% and 2.5% higher than the support vector machine and naive Bayes model.

As described above, according to an embodiment of the present disclosure, the stress level according to the emotional state may be more accurately classified by extracting the feature vector of the ECG signal and classifying the extracted feature vector using an ensemble model.

Meanwhile, 6 is a flowchart illustrating a mental stress classification method using an ensemble model according to an embodiment of the present disclosure.

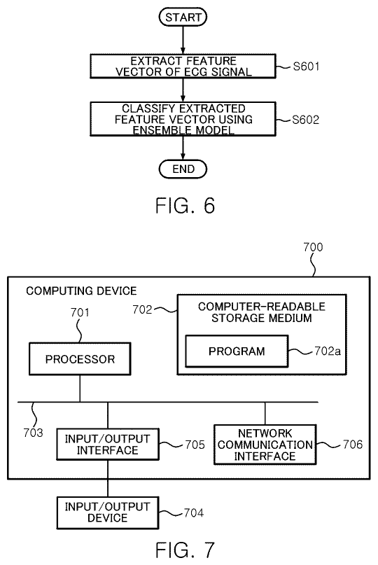

Hereinafter, a mental stress classification method (S600) using an ensemble model according to an embodiment of the present disclosure is described with reference to FIGS. 1 to 6. However, for simplification of the disclosure, descriptions given above with reference to FIGS. 1 to 5 are omitted.

Referring to FIGS. 1 to 6, the stress classification method (S600) using an ensemble model according to an embodiment of the present disclosure may start with an operation of extracting a feature vector of an ECG signal by the stress classification apparatus 110 (S601).

Thereafter, the stress classification apparatus 110 may classify the extracted feature vector using the ensemble model (S602). As described above, the classification unit 112 includes the ensemble model, and the ensemble model may be a mixed model of the support vector machine 112a and the naive Bayes 112b.

In addition, according to an embodiment of the present disclosure, the feature vector may include at least one of the R-S peak, R-R interval, and Q-T interval of the ECG signal, and the stress classification apparatus 110 may classify the ECG signal based on each feature vector, as described above.

In addition, according to an embodiment of the present disclosure, the aforementioned support vector machine 112a described above may be a pre-trained model that classifies each feature vector into one of multi-classes using the decision boundary.

In addition, the naive Bayes 112b may be a pre-trained model that classifies the stress index for the feature vector based on the preset contour plot in the class classified by the decision boundary obtained through the support vector machine.

In addition, as described above, the aforementioned contour plot may be obtained using the probability density function of the normal distribution for feature vectors belonging to each multi-class based on the ECG signal stored in the database 10 through a learning process.

Meanwhile, the database 10 may include a CLAS database storing ECG signals according to the test subject's emotions, and the ECG signals according to the test subject's emotions may include an emotional state by looking at landscape photos, an ECG signal that measures an emotional state after listening to classical music, an ECG signal that measures an emotional state after matching colors through visual stimulation, and an ECG signal that measures an emotional state after calculating the four arithmetic operations as described above.

According to an embodiment of the present disclosure, the mental stress classification apparatus 110 may remove noise by low-pass filtering the ECG signal and may use a Butterworth low-pass filter for the low-pass filtering as described above.

As described above, according to an embodiment of the present disclosure, the stress level according to an emotional state may be more accurately classified by extracting the feature vector of the ECG signal and classifying the extracted feature vector using the ensemble model.

FIG. 7 is a block diagram of a computing device 700 that may fully or partially implement the mental stress classification apparatus 110 according to an embodiment of the present disclosure.

As shown in FIG. 7, the computing device 700 includes at least one processor 701, a computer-readable storage medium 702, and a communication bus 703.

The processor 701 may cause the computing device 700 to operate according to the aforementioned embodiments. For example, the processor 701 may execute one or more programs stored in the computer-readable storage medium 702. The one or more programs may include one or more computer-executable instructions, which, when executed by the processor 701, may be configured to cause the computing device 700 to perform operations according to the embodiments.

The computer-readable storage medium 702 is configured to store computer-executable instructions or program code, program data, and/or other suitable forms of information. A program 702a stored in the computer-readable storage medium 702 includes a set of instructions executable by the processor 701. In an embodiment, the computer-readable storage medium 702 may include memory (volatile memory, such as random access memory, non-volatile memory, or an appropriate combination thereof), one or more magnetic disk storage devices, optical disk storage devices, flash memory devices, another type of storage medium that may be accessed by the computing device 700 and store desired information, or a suitable combination thereof.

The communication bus 703 interconnects various other components of the computing device 700 including the processor 701 and the computer-readable storage medium 702.

The computing device 700 may also include one or more input/output interfaces 705 providing an interface for one or more input/output devices 704 and one or more network communication interfaces 706. The input/output interface 705 and the network communication interface 706 are connected to the communication bus 703. The network may be one of a cellular network, such as global system for mobile communications (GSM), enhanced data rates for GSM evolution (EDGE), general packet radio service (GPRS), code division multiple access (CDMA), time division-CDMA (TD-CDMA), universal mobile telecommunications system (UMTS), long term evolution (LTE), or other cellular networks.

The input/output device 704 may be coupled to other components of the computing device 700 through input/output interface 705. As an example, the input/output devices 704 may include, but are not limited to, an input device, such as a pointing device (such as a mouse or trackpad), a keyboard, a touch input device (such as a touchpad or touch screen), a voice or sound input device, various types of sensor devices, and/or imaging devices and/or an output device, such as a display devices, a printer, a speaker, and/or a network card. For example, the input/output device 704 may be included within the computing device 700 as a component constituting the computing device 700 or may be connected to the computing device 700 as a separate device distinct from the computing device 700.

According to an embodiment of the present disclosure, stress levels according to emotional states may be more accurately classified by extracting feature vectors of ECG signals and classifying the extracted feature vectors using the ensemble model.

Meanwhile, the embodiments of the present disclosure may include a program for performing the methods described in this specification on a computer and a computer-readable recording medium including the program. The computer-readable recording medium may include program instructions, local data files, local data structures, etc., alone or in combination. The medium may be those specifically designed and configured for the present disclosure or may be those commonly available in the computer software field. Examples of computer-readable recording medium include magnetic medium, such as hard disks, floppy disks, and magnetic tapes, optical recording medium, such as CD-ROMs, DVDs, and hardware devices specifically configured to store and perform program instructions, such as ROM, RAM, flash memory, etc. Examples of the program may include not only machine language code, such as that generated by a compiler, but also high-level language code that may be executed by a computer using an interpreter or the like.

While the present disclosure has been particularly shown and described with reference to embodiments thereof, a person skilled in the art will understand that the invention is not limited to the disclosed embodiments but may be variously modified within the scope of the present disclosure. Therefore, the scope of the present disclosure should not be limited to the aforementioned embodiments but should be determined by all changes or modifications derived from the scope of the appended claims and equivalents of the following claims.

What is claimed is:

1. A mental stress classification apparatus using an ensemble model, the mental stress classification apparatus comprising:
a processor; and
a storage medium on which one or more programs configured to be executable by the processor are recorded,
wherein the processor is configured to
extract a feature vector of an electrocardiogram (ECG) signal, and
classify the extracted feature vector using the ensemble model,
wherein the ensemble model is a mixture model of a support vector machine and a naive Bayes.

2. The mental stress classification apparatus of claim 1, wherein
the feature vector includes at least one of an R-S peak, an R-R interval, and a Q-T interval of the ECG signal, and
the processor classifies each feature vector.

3. The mental stress classification apparatus of claim 1, wherein
the support vector machine is a pre-trained model that classifies the feature vector into one of multi-classes using a decision boundary, and
the naive Bayes is a pre-trained model that classifies a stress index for the feature vector based on a preset contour plot in the class classified by the decision boundary.

4. The mental stress classification apparatus of claim 3, wherein the contour plot is obtained using a probability density function of a normal distribution for feature vectors belonging to each of the multi-classes, based on an ECG signal stored in a database.

5. The mental stress classification apparatus of claim 4, wherein the database includes a cognitive load affect and stress (CLAS) database storing ECG signals according to a test subject's emotions.

6. The mental stress classification apparatus of claim 5, wherein the ECG signal according to the test subject's emotional state includes an ECG signal that measures an emotional state by looking at landscape photos, an ECG signal that measures an emotional state after listening to classical music, an ECG signal that measures an emotional state after matching colors through visual stimulation, and an ECG signal that measures an emotional state after calculating the four arithmetic operations.

7. The mental stress classification apparatus of claim 1, wherein
the processor is further configured to remove noise by low-pass filtering the ECG signal,
wherein the low-pass filter is a Butterworth low-pass filter.

8. A mental stress classification method using an ensemble model, the mental stress classification method comprising:
an extraction operation of extracting a feature vector of an electrocardiogram (ECG) signal; and
a classification operation of classifying the extracted feature vector using the ensemble model,
wherein the ensemble model is a mixture model of a support vector machine and a naive Bayes.

9. The mental stress classification method of claim 8, wherein
the feature vector includes at least one of an R-S peak, an R-R interval, and a Q-T interval of the ECG signal, and
in the classification operation, each feature vector is classified.

10. The mental stress classification method of claim 8, wherein
the support vector machine is a pre-trained model that classifies the feature vector into one of multi-classes using a decision boundary, and
the naive Bayes is a pre-trained model that classifies a stress index for the feature vector based on a preset contour plot in the class classified by the decision boundary.

11. The mental stress classification method of claim 10, wherein the contour plot is obtained using a probability density function of a normal distribution for feature vectors belonging to each of the multi-classes, based on an ECG signal stored in a database.

12. The mental stress classification method of claim 11, wherein the database includes a cognitive load affect and stress (CLAS) database storing ECG signals according to a test subject's emotions.

13. The mental stress classification method of claim 12, wherein the ECG signal according to the test subject's emotional state includes an ECG signal that measures an emotional state by looking at landscape photos, an ECG signal that measures an emotional state after listening to classical music, an ECG signal that measures an emotional state after matching colors through visual stimulation, and an ECG signal that measures an emotional state after calculating four arithmetic operations.

14. The mental stress classification method of claim 8, further comprising:
removing noise by low-pass filtering the ECG signal,
wherein the low-pass filter is a Butterworth low-pass filter.
